SEQUENCE LISTING (41 sequences)

SEQ ID NO: 1 - DNA, 1425 bp, synthetic construct
  source 1..1425: mol_type = other DNA
atgttgactgttggtttggccattgttgctgtcttggttatctgtgttactcacttgatttacagatggagaaacccaaagtgtaacggtatcttaccaccaggttccatgggtttcccattgatcggtgaaaccattcaattgatcatcccaggctacaccctcgacttgcctccattcatcaagaagagagttcaacgttacggtccaattttcagaaccaacctgttgggtagaccaatcattgtcactgctgatccagaaattaacaacttcatctaccaacaagaaggcagatctgttgaattatggtatcttgactccatttccaaggtcttcaagcaagacggtgactctcgtaccaccgctgggggtggtgtacacaagtacttgagatctatcaccttgaaccacttcggttccgaatccatcaaagaaaaattgttgccagacatccaaagattggtctccaaagttttccaccaatggtctaagcatccatccgttgaagtccaaagaggtaccttaaccatgttgtatgacttcaacgctgaaaaaatgttcggttacgatgctgaaaagtctagtgaaaacatctcagaatctttgatcactctagccgaaggtttcatggccatcccagttaacattccaggtactaagtacaacaagtgcttgaaagctcaaaagaagttgatcaacatgttcaagggtttagtcgctgaaagacaaagagctcgtgccggtgtcggtgccggtgatttcttggaccaagctgtgagagatattgaaaatgaacaattcttgactgatgaattcgttgtcaacttgatgtttggtatcttgttcgcttctggttctatctccggttctctgacattaatgtttaagttattggctgagaacccatctgttttgagagaattgactgaagaacacgaagctttcttgaagcaaaaggaaaaccctgaatctccaatcacctgggaagaatacaagtccatgactttcaccttgcaagtcattaacgaaactttaagattaagcaatgctttgccatttttgatgagaagaactgtcaaggatgtcgaaatcaagggttacactatcccagctggttggactgttatggttgcaaactctgctttgcatttggacccaaacacttttaaggacccattggactttaatccatggagatggaaggaccaagatcaatactccatttccaagaagttgatgccattcggtggtggtgccagacaatgtgctggtgctgactactctcgtgttttcatggctatcttcattcacactttggttaccaagtacagatggaagaaggttaagggtggtgaaatttccagatctccaattttgaagttcggtgatggtattcacattaccttctctcaaaaagcctga

SEQ ID NO: 2 - DNA, 1518 bp, synthetic construct
  source 1..1518: mol_type = other DNA
atggttgactctaacagtacccttttgtactcagtcttctccgttgtttccatcgttattgccattaagttctggttgaactctcgtaggatcagaaacttgcctccttctccaccaagcatcccaattattggtcacttacatttgttgaagccaccagttcacagatctttgcaagctttgtcccaaaagtatggtccaatcttctctttgaaattcggttccagattggttgtggtcgttacctctatggaaattgctgaagaatgtttcaccaagaacgatattgtttttgctaacagaccagattttgctgtctccaactgtttgtcctacaacaacactaccatcggtgctgctccatacggtgaccactggagaaagttgagaagagtctctgccctcgaaatcttgtcttctaatagattgcaaatgaacttgggtatcagaagagacgaaattcgtcgtttggtcaagaaactatcttctcacggtgacttcgctaaggtcaacttgaagtctatcgtcaaagaactgaccttaaacatcacaatgagaatggttgctggtaagcggtattacggtgaagaagaaaaggaagacgatgaagccagaactttccaagttttggtcgatgaattatttgaattcactttatcctcctacccaggtgatttcttgccaattttgaagtacatcgacatccaaggtttcatgaaaagagctagaaaattgatgaagaaggttgatgctttctggcaaggtttaattgacgaacaccgtacttacaagaacggtaacgttgaaatggaaaactccatgatttctcatttgttgaagttgcaagaatctcaaccagaatactacactgaagatattatcaagggtttgatcttgaccatgattttgggtggttccgataccactgctgttactatagaatgggctatgtctaatctgttgaaccacccatctgttatgaacaaggctagagccgaattggaatctcaaatcgaaactgacaagctagtcgaagaatccgacttgtctaagttgtcttacttgagatgtatcatcttagaaaccttgagattatacccagctggtccattgttgttgccacacagatcctcagctgactccactttgtccaagtacaacgtcccatctgacactatcttgttggttaacgcttgggccattcacagagatccaaccttgtgggatgacccaacttctttccgtccagaaagattcgaaaatagtgaaggtgaagctcacaagttcgtcatctctttcggtttgggcagaagagcttgcccaggtactggtatggcccatagagttttaggtttgacactaggtaccttcatccaatgtttcgaatgggacagaattgaaggtaaggaaattgacatggctgaaggtaagggtgtttccatgccaaaggctgagccattggaagcattgtgtaagccaagacacgtcgctatcaaggccatttcctcaaccttttga

SEQ ID NO: 3 - DNA, 30 bp, synthetic construct
  source 1..30: mol_type = other DNA
tttgccaacaatcgaaaccaaacatatatc

SEQ ID NO: 4 - DNA, 53 bp, synthetic construct
  source 1..53: mol_type = other DNA
caaatcttaaagtcatacattgcacgactaatataaagcagccgctaccaaac

SEQ ID NO: 5 - DNA, 53 bp, synthetic construct
  source 1..53: mol_type = other DNA
gtttggtagcggctgctttatattagtcgtgcaatgtatgactttaagatttg

SEQ ID NO: 6 - DNA, 59 bp, synthetic construct
  source 1..59: mol_type = other DNA
ggtactgttagagtcaaccatttattgtaatatgtgtgtttgtttggattattaagaag

SEQ ID NO: 7 - DNA, 53 bp, synthetic construct
  source 1..53: mol_type = other DNA
ccaaacaaacacacatattacaataaatggttgactctaacagtacccttttg

SEQ ID NO: 8 - DNA, 50 bp, synthetic construct
  source 1..50: mol_type = other DNA
ggtagattgggctacgtaaattcgatcaaaaggttgaggaaatggccttg

SEQ ID NO: 9 - DNA, 50 bp, synthetic construct
  source 1..50: mol_type = other DNA
aggccatttcctcaaccttttgatcgaatttacgtagcccaatctaccac

SEQ ID NO: 10 - DNA, 58 bp, synthetic construct
  source 1..58: mol_type = other DNA
acgcttgacatctactatatgtaagtatacggccccggcgccacttctataaaaggtc

SEQ ID NO: 11 - DNA, 58 bp, synthetic construct
  source 1..58: mol_type = other DNA
caatatcatgagaccttttatagaagtggcgccggggccgtatacttacatatagtag

SEQ ID NO: 12 - DNA, 55 bp, synthetic construct
  source 1..55: mol_type = other DNA
ggccaaaccaacagtcaacatgtttagttaattatagttcgttgaccgtatattc

SEQ ID NO: 13 - DNA, 59 bp, synthetic construct
  source 1..59: mol_type = other DNA
tttagaatatacggtcaacgaactataattaactaaacatgttgactgttggtttggcc

SEQ ID NO: 14 - DNA, 56 bp, synthetic construct
  source 1..56: mol_type = other DNA
cattaaagtaacttaaggagttaaatttatcaggctttttgagagaaggtaatgtg

SEQ ID NO: 15 - DNA, 58 bp, synthetic construct
  source 1..58: mol_type = other DNA
gatggtattcacattaccttctctcaaaaagcctgataaatttaactccttaagttac

SEQ ID NO: 16 - DNA, 59 bp, synthetic construct
  source 1..59: mol_type = other DNA
cacagtgatatgcatatgggagatggagatgatacctgcgaaaagccaattagtgtgat

SEQ ID NO: 17 - DNA, 59 bp, synthetic construct
  source 1..59: mol_type = other DNA
catagcaatctaatctaagttttaattacaaaatgtcatctaatggtcaatctccatcc

SEQ ID NO: 18 - DNA, 59 bp, synthetic construct
  source 1..59: mol_type = other DNA
cgaactcttgcatcttacgatacctgagtattcccacagttagtgatcccccacacacc

SEQ ID NO: 19 - DNA, 57 bp, synthetic construct
  source 1..57: mol_type = other DNA
gcagaaaagactaataattcttagttaaaagcacctattgacccaaggttttggcag

SEQ ID NO: 20 - DNA, 59 bp, synthetic construct; identical to SEQ ID NO: 17
  source 1..59: mol_type = other DNA

SEQ ID NO: 21 - DNA, 59 bp, synthetic construct
  source 1..59: mol_type = other DNA
gataaagcacttagtatcacactaattggcttttcgcaggtatcatctccatctcccat

SEQ ID NO: 22 - DNA, 57 bp, synthetic construct
  source 1..57: mol_type = other DNA
cgaattcactgccaaaaccttgggtcaataggtgcttttaactaagaattattagtc

SEQ ID NO: 23 - DNA, 59 bp, synthetic construct
  source 1..59: mol_type = other DNA
aaacattttgaagctatggtgtgtgggggatcactaactgtgggaatactcaggtatcg

SEQ ID NO: 24 - DNA, 58 bp, synthetic construct
  source 1..58: mol_type = other DNA
aattatatgatataaaggcgcctggccgattaagcaaggattttcttaacttcttcgg

SEQ ID NO: 25 - DNA, 59 bp, synthetic construct
  source 1..59: mol_type = other DNA
taattatatgatataaaggcgcctggccgattaagcaaggattttcttaacttcttcgg

SEQ ID NO: 26 - DNA, 42 bp, synthetic construct
  source 1..42: mol_type = other DNA
gactataatattatgcatataggatataccaaaaattctctc

SEQ ID NO: 27 - DNA, 802 bp, synthetic construct
  source 1..802: mol_type = other DNA
tttgccaacaatcgaaaccaaacatatatcttaaaatacaccactttctcaaataaattcgttaaataacggtgtgttgaaatgtttaccgtaacttgtaacagctctaacaactcatacctgctatgtactgattccaagaaaaaaaaaatggaaggtcgggatgagcatatacaagcactaagaagaacaatacagaactctacacggtattattgtgctacaagctcgagtaaaaccgagtgttttgacgatactaacgttgttaagaaagtaacttgttatcaaactcattaccaacttgtgattaattggtgaataatatgataattgtcgaaattccattgttggtaaagcctataatattatgtatacagattatactagaaattctctcgagaatataagaatccccaaaattgaatcggtatttctacatactaatattaccattacttctcctttcgttttatatgtttcattcctattacattatcgatctttgcatttcagcttccattatatttgatgtctgttttatgtccccacgttacaccgcatgtgacagtatactagtaacatgagtgctaccgaatagatgacattttagactttcattccaacaacttggttgacagaatgttacgtaccctatatctaatctatatgaggcctgaatctaactgaaaggtggaatttcagtaatttatcaagctttaataagtttgggtagtttaactgtgcaaaaaggtatttaccttacatactgaatcttgtctgtttggtagcggctgctttatat

SEQ ID NO: 28 - DNA, 759 bp, synthetic construct
  source 1..759: mol_type = other DNA
tagtcgtgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaataa

SEQ ID NO: 29 - DNA, 230 bp, synthetic construct
  source 1..230: mol_type = other DNA
tcgaatttacgtagcccaatctaccacttttttttttcattttttaaagtgttatacttagttatgctctaggataatgaactacttttttttttttttttttactgttatcataaatatatataccttattgttgtttgcaaccgtcggttaattccttatcaaggttccccaagttcggatcattaccatcgtaacaatatcatgagaccttttatagaagtggcgcc

SEQ ID NO: 30 - DNA, 560 bp, synthetic construct
  source 1..560: mol_type = other DNA
ggggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaac

SEQ ID NO: 31 - DNA, 410 bp, synthetic construct
  source 1..410: mol_type = other DNA
taaatttaactccttaagttactttaatgatttagtttttattattaataattcatgctcatgacatctcatatacacgtttataaaacttaaatagattgaaaatgtattaaagattcctcagggattcgatttttttggaagtttttgtttttttttccttgagatgctgtagtatttgggaacaattatacaatcgaaagatatatgcttacattcgaccgttttagccgtgatcattatcctatagtaacataacctgaagcataactgacactactatcatcaatacttgtcacatgagaactctgtgaataattaggccactgaaatttgatgcctgaaggaccggcatcacggattttcgataaagcacttagtatcacactaattggcttttcgcaggtatc

SEQ ID NO: 32 - DNA, 430 bp, synthetic construct
  source 1..430: mol_type = other DNA
tttgtaattaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatggtgtgtgggggatcact

SEQ ID NO: 33 - DNA, 840 bp, synthetic construct
  source 1..840: mol_type = other DNA
ctattgacccaaggttttggcagtgaattcgaaagatggattagtcaaagagaaaccaccatcaacaaccaagttcaaaccagacacgtatttggcttcatcagaacccaaataaacagcagctttagcaacatcttcagcttccatagcagcacctttcaaattaccggacatggagatgacgtattccatcatctttttctcttcctcgttcatagatggcaacaacattctagtaacggtagcgaatggagaaatgcagttgactctaataccatgtttacccaattcaacagccaaggtcttagtcataccaactatagcatgttttgtagcagagtaagctggggtgttaccagaacaaataacagattccatagaagaggtgaacaggatacaacctttcttagctggaatcataactctggcagcatgtttagcacccataaaagtaccgaaaacattgacgtcgaaaacgtgtctgaattccttcatatcagtggtttcagtcttagaagtgacgttaccacaaataccagcgttgttgaacatgatgtccaaagtaccatacttgtcaatggcgaagttaacagcgttttcaatatcggtttcgtcagtaacatcacaatgcaagtaggagatctcacgaccaatagaacccaattctttacacaaggtgtcagccaaatcatctctaacatcagcgataataaccttggcaccttctctagtgaataatctaacagtagattcacccaaaccagaagcaccaccagtaatcaaagcaaccttaccatccaatctttttggaatggatggagattgaccattagatgacat

SEQ ID NO: 34 - DNA, 392 bp, synthetic construct
  source 1..392: mol_type = other DNA
atctccatctcccatatgcatatcactgtggtcgtggcccgcatttccttccatcttgtgattcatgctatccatcttttttgagtatccaattaacgaagacgttaccagctgattgaaggttctcaaagtgactgtactccatgttttcttatcatccatgtagttatttttcaaactgcaaattcaagaaaaagccacgcgtgtgcaccttttttttccccttccagtgcattatgcaatagacagcacgagtctttgaaaaagtaacttataaaactgtatcaatttttaaacctaaatagattcataaactattcgttaatataaagtgttctaaactatgatgaaaaaataagcagaaaagactaataattcttagttaaaagcac

SEQ ID NO: 35 - DNA, 1512 bp, synthetic construct
  source 1..1512: mol_type = other DNA
aactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaa

SEQ ID NO: 36 - DNA, 846 bp, synthetic construct
  source 1..846: mol_type = other DNA
tcggccaggcgcctttatatcatataattaagacacaaaaggataaaacaaaggtgttaactattctgcatactcactatcgtaaactgtcctgcaaatcgtgtaaatatgtatttcattttttttgcagtgaaaaaaggcatgtaaaataccgcatcaagtaactctactccgcctgtggtttcaagactaacggcttgagacaaaatgggaagaaatgattgcagaaaagccatatgtgtaatagcaaaaagctggatagtgcttaccagatgtttaccttaatttcttggtgaattagagaagtacagaagttttactattaatcccaccatagaaatttgtataggaaagtagtttattggagttattggatatactgtgtaaactatttcttgaaattgtaatcttaagatgctcttcttattctattaaaaatagaaaatgattttcatatttatttatttatttatattttggcattactcttcatcatttttttccctctaagaagcttcctttctttttataaggataacaaaaccaaaaggaatattgggtcagatgaatggacgcgaatgcaagacagaagtccaaatcacgtcaagacaaagaaagaaagaaagaaaaactaacacattaatgtagttttaaaatttcaaatccgaacaacagagcatagggtttcgcaaacaaacttaaatatatgctagaataaaaattaactatcatctattgactagtattcatatatgacgtaataaaatgaaatgtagattcattttgtagattcctatatcctcagagagaatttttggtatatcctatatgcataatattatagtc

SEQ ID NO: 37 - DNA, 9724 bp, synthetic construct
  source 1..9724: mol_type = other DNA
tttgccaacaatcgaaaccaaacatatatcttaaaatacaccactttctcaaataaattcgttaaataacggtgtgttgaaatgtttaccgtaacttgtaacagctctaacaactcatacctgctatgtactgattccaagaaaaaaaaaatggaaggtcgggatgagcatatacaagcactaagaagaacaatacagaactctacacggtattattgtgctacaagctcgagtaaaaccgagtgttttgacgatactaacgttgttaagaaagtaacttgttatcaaactcattaccaacttgtgattaattggtgaataatatgataattgtcgaaattccattgttggtaaagcctataatattatgtatacagattatactagaaattctctcgagaatataagaatccccaaaattgaatcggtatttctacatactaatattaccattacttctcctttcgttttatatgtttcattcctattacattatcgatctttgcatttcagcttccattatatttgatgtctgttttatgtccccacgttacaccgcatgtgacagtatactagtaacatgagtgctaccgaatagatgacattttagactttcattccaacaacttggttgacagaatgttacgtaccctatatctaatctatatgaggcctgaatctaactgaaaggtggaatttcagtaatttatcaagctttaataagtttgggtagtttaactgtgcaaaaaggtatttaccttacatactgaatcttgtctgtttggtagcggctgctttatattagtcgtgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatattacaataaatggttgactctaacagtacccttttgtactcagtcttctccgttgtttccatcgttattgccattaagttctggttgaactctcgtaggatcagaaacttgcctccttctccaccaagcatcccaattattggtcacttacatttgttgaagccaccagttcacagatctttgcaagctttgtcccaaaagtatggtccaatcttctctttgaaattcggttccagattggttgtggtcgttacctctatggaaattgctgaagaatgtttcaccaagaacgatattgtttttgctaacagaccagattttgctgtctccaactgtttgtcctacaacaacactaccatcggtgctgctccatacggtgaccactggagaaagttgagaagagtctctgccctcgaaatcttgtcttctaatagattgcaaatgaacttgggtatcagaagagacgaaattcgtcgtttggtcaagaaactatcttctcacggtgacttcgctaaggtcaacttgaagtctatcgtcaaagaactgaccttaaacatcacaatgagaatggttgctggtaagcggtattacggtgaagaagaaaaggaagacgatgaagccagaactttccaagttttggtcgatgaattatttgaattcactttatcctcctacccaggtgatttcttgccaattttgaagtacatcgacatccaaggtttcatgaaaagagctagaaaattgatgaagaaggttgatgctttctggcaaggtttaattgacgaacaccgtacttacaagaacggtaacgttgaaatggaaaactccatgatttctcatttgttgaagttgcaagaatctcaaccagaatactacactgaagatattatcaagggtttgatcttgaccatgattttgggtggttccgataccactgctgttactatagaatgggctatgtctaatctgttgaaccacccatctgttatgaacaaggctagagccgaattggaatctcaaatcgaaactgacaagctagtcgaagaatccgacttgtctaagttgtcttacttgagatgtatcatcttagaaaccttgagattatacccagctggtccattgttgttgccacacagatcctcagctgactccactttgtccaagtacaacgtcccatctgacactatcttgttggttaacgcttgggccattcacagagatccaaccttgtgggatgacccaacttctttccgtccagaaagattcgaaaatagtgaaggtgaagctcacaagttcgtcatctctttcggtttgggcagaagagcttgcccaggtactggtatggcccatagagttttaggtttgacactaggtaccttcatccaatgtttcgaatgggacagaattgaaggtaaggaaattgacatggctgaaggtaagggtgtttccatgccaaaggctgagccattggaagcattgtgtaagccaagacacgtcgctatcaaggccatttcctcaaccttttgatcgaatttacgtagcccaatctaccacttttttttttcattttttaaagtgttatacttagttatgctctaggataatgaactacttttttttttttttttttactgttatcataaatatatataccttattgttgtttgcaaccgtcggttaattccttatcaaggttccccaagttcggatcattaccatcgtaacaatatcatgagaccttttatagaagtggcgccggggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaacatgttgactgttggtttggccattgttgctgtcttggttatctgtgttactcacttgatttacagatggagaaacccaaagtgtaacggtatcttaccaccaggttccatgggtttcccattgatcggtgaaaccattcaattgatcatcccaggctacaccctcgacttgcctccattcatcaagaagagagttcaacgttacggtccaattttcagaaccaacctgttgggtagaccaatcattgtcactgctgatccagaaattaacaacttcatctaccaacaagaaggcagatctgttgaattatggtatcttgactccatttccaaggtcttcaagcaagacggtgactctcgtaccaccgctgggggtggtgtacacaagtacttgagatctatcaccttgaaccacttcggttccgaatccatcaaagaaaaattgttgccagacatccaaagattggtctccaaagttttccaccaatggtctaagcatccatccgttgaagtccaaagaggtaccttaaccatgttgtatgacttcaacgctgaaaaaatgttcggttacgatgctgaaaagtctagtgaaaacatctcagaatctttgatcactctagccgaaggtttcatggccatcccagttaacattccaggtactaagtacaacaagtgcttgaaagctcaaaagaagttgatcaacatgttcaagggtttagtcgctgaaagacaaagagctcgtgccggtgtcggtgccggtgatttcttggaccaagctgtgagagatattgaaaatgaacaattcttgactgatgaattcgttgtcaacttgatgtttggtatcttgttcgcttctggttctatctccggttctctgacattaatgtttaagttattggctgagaacccatctgttttgagagaattgactgaagaacacgaagctttcttgaagcaaaaggaaaaccctgaatctccaatcacctgggaagaatacaagtccatgactttcaccttgcaagtcattaacgaaactttaagattaagcaatgctttgccatttttgatgagaagaactgtcaaggatgtcgaaatcaagggttacactatcccagctggttggactgttatggttgcaaactctgctttgcatttggacccaaacacttttaaggacccattggactttaatccatggagatggaaggaccaagatcaatactccatttccaagaagttgatgccattcggtggtggtgccagacaatgtgctggtgctgactactctcgtgttttcatggctatcttcattcacactttggttaccaagtacagatggaagaaggttaagggtggtgaaatttccagatctccaattttgaagttcggtgatggtattcacattaccttctctcaaaaagcctgataaatttaactccttaagttactttaatgatttagtttttattattaataattcatgctcatgacatctcatatacacgtttataaaacttaaatagattgaaaatgtattaaagattcctcagggattcgatttttttggaagtttttgtttttttttccttgagatgctgtagtatttgggaacaattatacaatcgaaagatatatgcttacattcgaccgttttagccgtgatcattatcctatagtaacataacctgaagcataactgacactactatcatcaatacttgtcacatgagaactctgtgaataattaggccactgaaatttgatgcctgaaggaccggcatcacggattttcgataaagcacttagtatcacactaattggcttttcgcaggtatcatctccatctcccatatgcatatcactgtggtcgtggcccgcatttccttccatcttgtgattcatgctatccatcttttttgagtatccaattaacgaagacgttaccagctgattgaaggttctcaaagtgactgtactccatgttttcttatcatccatgtagttatttttcaaactgcaaattcaagaaaaagccacgcgtgtgcaccttttttttccccttccagtgcattatgcaatagacagcacgagtctttgaaaaagtaacttataaaactgtatcaatttttaaacctaaatagattcataaactattcgttaatataaagtgttctaaactatgatgaaaaaataagcagaaaagactaataattcttagttaaaagcacctattgacccaaggttttggcagtgaattcgaaagatggattagtcaaagagaaaccaccatcaacaaccaagttcaaaccagacacgtatttggcttcatcagaacccaaataaacagcagctttagcaacatcttcagcttccatagcagcacctttcaaattaccggacatggagatgacgtattccatcatctttttctcttcctcgttcatagatggcaacaacattctagtaacggtagcgaatggagaaatgcagttgactctaataccatgtttacccaattcaacagccaaggtcttagtcataccaactatagcatgttttgtagcagagtaagctggggtgttaccagaacaaataacagattccatagaagaggtgaacaggatacaacctttcttagctggaatcataactctggcagcatgtttagcacccataaaagtaccgaaaacattgacgtcgaaaacgtgtctgaattccttcatatcagtggtttcagtcttagaagtgacgttaccacaaataccagcgttgttgaacatgatgtccaaagtaccatacttgtcaatggcgaagttaacagcgttttcaatatcggtttcgtcagtaacatcacaatgcaagtaggagatctcacgaccaatagaacccaattctttacacaaggtgtcagccaaatcatctctaacatcagcgataataaccttggcaccttctctagtgaataatctaacagtagattcacccaaaccagaagcaccaccagtaatcaaagcaaccttaccatccaatctttttggaatggatggagattgaccattagatgacattttgtaattaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatggtgtgtgggggatcactaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaatcggccaggcgcctttatatcatataattaagacacaaaaggataaaacaaaggtgttaactattctgcatactcactatcgtaaactgtcctgcaaatcgtgtaaatatgtatttcattttttttgcagtgaaaaaaggcatgtaaaataccgcatcaagtaactctactccgcctgtggtttcaagactaacggcttgagacaaaatgggaagaaatgattgcagaaaagccatatgtgtaatagcaaaaagctggatagtgcttaccagatgtttaccttaatttcttggtgaattagagaagtacagaagttttactattaatcccaccatagaaatttgtataggaaagtagtttattggagttattggatatactgtgtaaactatttcttgaaattgtaatcttaagatgctcttcttattctattaaaaatagaaaatgattttcatatttatttatttatttatattttggcattactcttcatcatttttttccctctaagaagcttcctttctttttataaggataacaaaaccaaaaggaatattgggtcagatgaatggacgcgaatgcaagacagaagtccaaatcacgtcaagacaaagaaagaaagaaagaaaaactaacacattaatgtagttttaaaatttcaaatccgaacaacagagcatagggtttcgcaaacaaacttaaatatatgctagaataaaaattaactatcatctattgactagtattcatatatgacgtaataaaatgaaatgtagattcattttgtagattcctatatcctcagagagaatttttggtatatcctatatgcataatattatagtc

SEQ ID NO: 38 - DNA, 30 bp, synthetic construct; identical to SEQ ID NO: 3
  source 1..30: mol_type = other DNA

SEQ ID NO: 39 - DNA, 25 bp, synthetic construct
  source 1..25: mol_type = other DNA
tcaaaaggttgaggaaatggccttg

SEQ ID NO: 40 - DNA, 29 bp, synthetic construct
  source 1..29: mol_type = other DNA
ctagtgaaaacatctcagaatctttgatc

SEQ ID NO: 41 - DNA, 22 bp, synthetic construct
  source 1..22: mol_type = other DNA
cgtcgaaaacgtgtctgaattc

(19) United States
(12) Patent Application Publication
(10) Pub. No.: US 2025/0236894 A1
(43) Pub. Date: Jul. 24, 2025

(54) APPLICATION OF TRANSGENIC SACCHAROMYCES CEREVISIAE ENGINEERING BACTERIA OF HEMSLEYA CHINENSIS CYTOCHROME OXIDASE IN PREPARATION OF CUCURBITACIN INTERMEDIATE

(71) Applicants: Yunnan Agricultural University, Kunming City (CN); Yunnan Yunke Characteristic Plant Extraction Laboratory Co., Ltd., Kunming City (CN)

(72) Inventors: Guanghui ZHANG, Kunming City (CN); Chunli HUANG, Kunming City (CN); Shengchao YANG, Kunming City (CN); Bing HAO, Kunming City (CN); Geng CHEN, Kunming City (CN); Yanyu SHU, Kunming City (CN); Zhiyuan LI, Kunming City (CN); Xiaolin FENG, Kunming City (CN); Simei HE, Kunming City (CN); Yingchun LU, Kunming City (CN)

(73) Assignees: Yunnan Agricultural University, Kunming City, YN (CN); Yunnan Yunke Characteristic Plant Extraction Laboratory Co., Ltd., Kunming City (CN)

(21) Appl. No.: 19/014,203

(22) Filed: Jan. 8, 2025

(30) Foreign Application Priority Data
Jan. 19, 2024 (CN) 202410119212.5

(51) Int. Cl. C12P 5/00; C12N 15/81; C12P 7/42

(52) U.S. Cl. CPC C12P 5/007; C12N 15/81; C12P 7/42; C12N 2800/101

(57) ABSTRACT

The invention provides a Hemsleya chinensis cytochrome oxidase HcCYP87D19 gene, and the sequence of the gene is shown in SEQ NO: 1. This invention constructs a transgenic Saccharomyces cerevisiae engineering bacteria co-transfected with Hemsleya cathayensis cytochrome oxidase HcCYP87D19 and HcCYP81Q58, the bacteria can produce a variety of cucurbitacin intermediates including 11-Carbonyl-cucurbita-5,23-diene-3β,16a,20,25-tetrol (6), 11-Carbonyl-cucurbita-5,23-diene-3β,16α,20,23-tetrol (6b), 11-Carbonyl-cucurbita-5,23-diene-16α,20,25-triol-3-one (6a), 11-Carbonyl-cucurbita-5,24-diene-16α,20,23-triol-3-one (6c), it provides a source for cucurbitacin production.

This instant application contains a Sequence Listing which has been submitted electronically in XML file format and is hereby incorporated by reference in its entirety. Said XML Copy, created on Jan. 8, 2025, is named Seqlisting.xml and is 56000 bytes in size.

### TECHNICAL FIELD

The invention belongs to the field of biotechnology, in particular to an application of a Hemsleya chinensis cytochrome oxidase gene HcCYP87D19 and its transgenic engineering bacteria in a preparation of cucurbitacin intermediates.

### BACKGROUND ART

Cucurbitacin is a highly oxidized tetracyclic triterpenoid compound with cucurbitane as the basic skeleton, it has high medicinal value, especially in anti-tumor. Although it has high anti-tumor activity, low therapeutic index, and non-specificity, its toxicity greatly hinders the clinical application and biological research of cucurbitacins. Moreover, the structure of triterpenoids is relatively complex, and the content in plants is low and often mixed with a variety of components with similar structures, which is difficult to obtain by extraction and separation or chemical synthesis. Only a small fraction of cucurbitacins have been developed as drugs (CuB, dihydrocucurbitacin I, etc.). More importantly, the complete biosynthetic pathway for the synthesis of cucurbitacin triterpenoids by modifying their skeletons remains unclear, the lack of this production pathway limits the utility and biological research of this highly oxidized compound as a clinical candidate drug, studies have shown that impurities in cucurbitacin preparations may be the cause of toxicity. Therefore, the synthetic biology strategy based on the elucidation of biosynthetic pathways seems to be a more promising, sustainable, and alternative approach.

Synthetic biology plays an important role in the production of pharmaceutical monomers. The use of synthetic biology to achieve a heterologous synthesis of cucurbitacin monomers is one of the most important strategies. In recent years, more and more medicinal active ingredients have been successfully biosynthesized in microorganisms, such as paclitaxel precursors, artemisinin precursors, artemisinic acid, ginsenosides, and their precursors. However, cucurbitacin biosynthesis is currently limited to the cucurbitadienol precursor (Cuol), which is mainly due to the lack of knowledge of its biosynthetic pathway, at the same time, there are few systems for the synthesis of cucurbitacin monomers and intermediates by using heterologous hosts, and there is no report on the production of cucurbitacin monomers and intermediates by metabolic engineering, the tubers of Hemsleya chinensis plants are rich in cucurbitacins, in particular, the dihydrocucurbitacin extracted from the tubers of this plants (a mixture of dihydrocucurbitacin I and dihydrocucurbitacin II) is a raw material for the production of Chinese medicines, such as dihydrocucurbitacin tablets (traditional Chinese medicine protection varieties) and dihydrocucurbitacin capsules. The drug has the functions of clearing heat and detoxifying, antibacterial, and anti-inflammatory, it is mainly used for the treatment of bacillary dysentery, enteritis, bronchitis, acute tonsillitis, and other diseases.

CYP450 is an ancient supergene family and the largest enzyme family in plant metabolism, CYP450 has a wide range of catalytic activity, it can insert an oxygen atom into a hydrophobic molecule in a biochemical reaction to obtain higher activity or hydrophilicity, also known as mixed-function oxidase (MFO). The most common reaction is the monooxygenase reaction, its catalytic carbon skeleton modification of triterpenes significantly promotes the structural diversity of triterpenes.

Studies have shown that cucurbitacin biosynthesis starts from the cyclization of 2,3-oxidosqualene to form cucurbitadienol (Cuol), which is catalyzed by cucurbitadienol synthase (CBS) of the oxidized squalene cyclase (OSCs) family. Cucurbitadienol is catalyzed by cytochrome P450 and is oxidized at specific sites by introducing hydroxyl, carboxyl, or epoxy groups to produce a variety of tetracyclic triterpenoids, the gene function of HcCYP81Q58 has been verified and patented, the patented strain Cuol-04-1 proves that HcCYP81Q58 can catalyze the C25 hydroxylation and C23 hydroxylation of 11-carbonyl-20β-hydroxy-Cuol to form 11-Carbonyl-cucurbita-5,23-diene-3β,20,25-triol (5), 11-Carbonyl-cucurbita-5,24-diene-38,20,23-triol (5b). Then, the C3 position is ketonized by HCSDR34 to form 11-Carbonyl-cucurbita-5,23-diene-20,25-diol-3β-one (5a), 11-Carbonyl-cucurbita-5,24-diene-20,23-diol-3β-one (5c).

Cucurbitacins are highly oxidized and have low content in plants, due to limited utilization, the extraction and purification process is complex and time-consuming, which requires a lot of manpower and material resources, at present, the development of related drugs is mainly prepared by extraction from plants. Nowadays, there are still many challenges in the chemical synthesis of cucurbitacins, especially the key intermediate compounds of cucurbitacin, the development of synthetic biology and metabolic engineering discloses a new opportunity for the efficient heterologous synthesis of tetracyclic triterpenoids. As a eukaryotic expression system, Saccharomyces cerevisiae has a clear genetic background and a mature genetic modification strategy. The endogenous mevalonate (MVA) pathway can disclose the required precursors for the synthesis of more terpenoids, which is conducive to the synthesis of terpenoids. Therefore, it is very important to establish and optimize Saccharomyces cerevisiae engineering bacteria for efficient production of cucurbitacin intermediates in the study of cucurbitacin biosynthesis pathway and possible future production. The catalytic steps of the cucurbitacin biological pathway are long and complex, and there is a lack of intermediates, therefore, there is an urgent need for genetic engineering bacteria that can produce high-yield cucurbitacin precursors and intermediates.

### SUMMARY OF THE INVENTION

In order to fill the gap in the existing technology, the invention aims to disclose an application of a Hemsleya chinensis cytochrome oxidase HcCYP87D19 and its transgenic engineering bacteria in a preparation of cucurbitacin intermediates. Specifically, the invention discloses the following technical solution:

In a first aspect, the invention discloses a Hemsleya chinensis cytochrome oxidase HcCYP87D19, a sequence of the gene is shown in SEQ NO: 1.

In a second aspect, the invention discloses an application of the above-mentioned Hemsleya chinensis cytochrome oxidase HcCYP87D19 in a preparation of cucurbitacin intermediates.

In an embodiment, the application is to co-transfect the cytochrome oxidase HcCYP87D19 and an HCCYP81Q58 into genetic engineering bacteria.

In an embodiment, the cucurbitacin intermediates comprise 11-Carbonyl-cucurbita-5,23-diene-3β,16a,20,25-tetrol(6), 11-Carbonyl-cucurbita-5,23-diene-3β,16α,20,23-tetrol(6b), 11-Carbonyl-cucurbita-5,23-diene-16α,20,25-triol-3-one(6a), 11-Carbonyl-cucurbita-5,24-diene-16α,20,23-triol-3-one(6c).

In a third aspect, the invention discloses an expression vector, which contains a promoter, HcCYP81Q58, HcCYP87D19, HcSDR34, LEU2, and a terminator.

In an embodiment, the promoter consists of GPMp, TEF2p, and TEF1p elements; the terminator consists of IDP1t, TDH2t and ENO2t.

In a fourth aspect, the invention discloses a transgenic Saccharomyces cerevisiae engineering bacteria, and the transgenic Saccharomyces cerevisiae engineering bacteria express the above-mentioned expression vector.

In a fifth aspect, the invention discloses a method for constructing the above-mentioned transgenic Saccharomyces cerevisiae engineering bacteria, the method comprises transfecting the above-mentioned expression vector into Saccharomyces cerevisiae.

In a sixth aspect, the invention discloses an application of the above-mentioned Hemsleya chinensis cytochrome oxidase HcCYP87D19, expression vector, and transgenic Saccharomyces cerevisiae engineering bacteria in a preparation of cucurbitacin intermediate products.

In the sixth aspect, the invention discloses an application of the above-mentioned method for constructing transgenic Saccharomyces cerevisiae engineering bacteria in the preparation of cucurbitacin intermediates.

Compared with the existing technology, the beneficial effect of the invention is reflected in:

The invention discloses a transgenic Saccharomyces cerevisiae engineering strain Coul-05-1 containing Hemsleya chinensis cytochrome oxidase HcCYP81Q58 and HcCYP87D19, which is used to synthesize 11-Carbonyl-cucurbita-5,23-diene-3β,16a,20,25-tetrol(6), 11-Carbonyl-cucurbita-5,23-diene-3β,16α,20,23-tetrol(6b), 11-Carbonyl-cucurbita-5,23-diene-16α,20,25-triol-3-one(6a), 11-Carbonyl-cucurbita-5,24-diene-16α,20,23-triol-3-one (6c) by bioengineering method, furthermore, it lays a foundation for the study of the regulation of dihydrocucurbitacin biosynthesis and provides a preliminary basis for the future bio-industrial production of cucurbitacins.

### BRIEF DESCRIPTION OF DRAWINGS

FIG. 1 is a schematic diagram of the construction of transgenic Saccharomyces cerevisiae Coul-05-1;

FIG. 2 is a synthesis pathway of the HcCYP87D19, it catalyzes the conversion of 5, 5b to 6, 6b;

FIG. 3 is an HPLC and LC-Ms detection of the CYPHC87D19 products. The upper figures are the HPLC detection results of compound 6 and compound 6a of HcCYP87D19 products, as well as the LC-MS mass spectra of compound 6 (M+Na+511 corresponding to 488) and compound 6a (M+Na+509 corresponding to 486). The lower figures are the HPLC detection results of compound 6b and compound 6c;

FIG. 4 is an NMR spectra of HcCYP87D19 product compound 6 (FIG. 4A, FIG. 4B), compound 6a (FIG. 4C, FIG. 4D), and compound 6c (FIG. 4E, FIG. 4F).

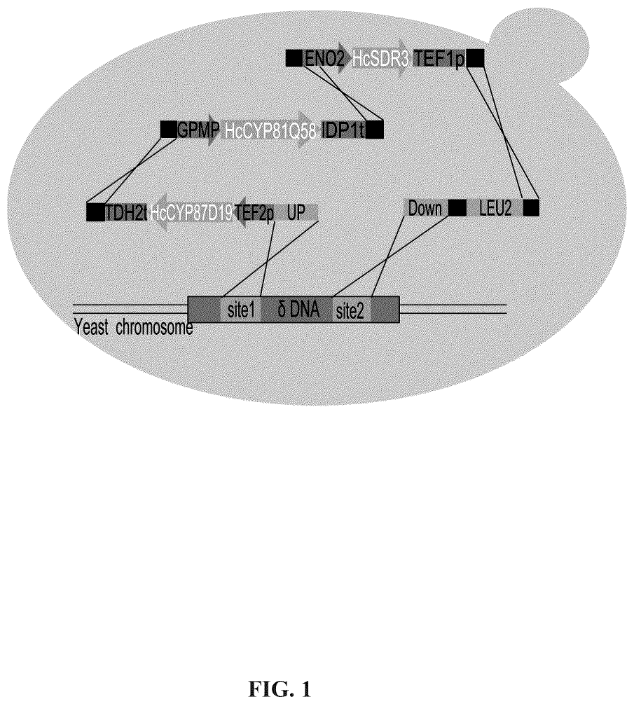

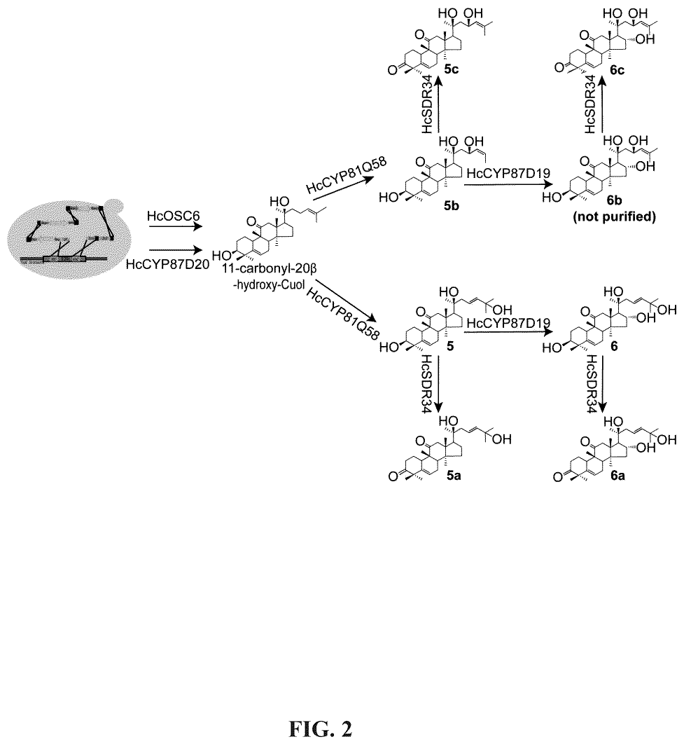

### DETAILED DESCRIPTION OF THE EMBODIMENTS

The following will explain the technical solution of the invention in combination with the embodiments. Technicians in this field will understand that the following embodiments are only used to illustrate the invention and should not be regarded as limitations to the scope of the invention. If the specific technology or conditions are not specified in the embodiment, it shall be carried out by the technology or conditions described in the literature in this field or accordance with the product description. Reagents or instruments used that do not specify the manufacturer are conventional products that can be obtained through commercial purchase.

### Embodiment 1: Cloning and Optimization of HcCYP87D19

Saccharomyces cerevisiae Genomic DNA Extraction

Saccharomyces cerevisiae BY4742 plaque is selected and cultured in YPD liquid medium (Formula: 1% Yeast Extract, 2% Peptone, 2% Dextrose) at the conditions of 30° C., 200 rpm, 24 h, 10000 g, 5 min, the bacteria are collected in a 1.5 ml centrifuge tube and washed twice with water. The strain is resuspended in the yeast wall-breaking solution (25 ul yeast wall-breaking enzyme, 470 ul sorbitol buffer, 5 ul B-ME), incubated at 30° C. for 1 h, and then centrifuged. The strain is recentrifuged with 500 ul TENTS buffer (10 mMTris-HCl, pH 7.5; 1 mM EDTA, pH 8.0:100 mM NaAc: 2% triton-100; 1% SDS), then it is in a 0° C. water bath for 1 h; it is extracted 2 times by phenol/chloroform; the supernatant is added with 3 times the volume of EtOH, 1/10 times the volume of 3M NaAc, and then it is placed in the refrigerator at 20° C. for 2 h t the conditions of 1300 g, 4° C., it is centrifuged for 10 min, the supernatant is removed, the precipitate is precipitated twice with 70% EtOH lotion and dried, the precipitate is dissolved in double distilled water and stored at −20° C. to obtain Saccharomyces cerevisiae genomic DNA.

### (2) Codon Optimization of HcCYP87D19 and HcCYP81Q58 Sequences

HcCYP87D19 and HcCYP81Q58 are obtained from the genome sequencing data of Hemsleya chinensis, then they are synthesized by Wuhan Jinkairui Bioengineering Co., Ltd. according to the optimized sequence of Saccharomyces cerevisiae codon. The nucleotide sequences of the optimized HcCYP87D19 and HcCYP81Q58 are shown in SEQ ID NO.1 and SEQ ID NO.2, respectively.

### Embodiment 2: Construction of Expression Vector (Construction of Gene Expression Cassette)

First, each promoter, gene, terminator, nutrient selection marker, and homologous arm is amplified by PCR using Q5® High-Fidelity DNA Polymerase (NEB: M0491). PCR reaction system is 95° C., 3 min; 95° C., 15S, 58° C., 2 min, 72° C., 1 min, 35 cycles; 72° C., 5 min.

Secondly, after the PCR amplification is completed, electrophoresis is carried out, and the target band is recovered after confirming the success of the amplification. The EasyPure Quick Gel Extraction Kit of Beijing All Gold Biotechnology Co., Ltd. is used for target gene recovery. After recovery, the recovery concentration is measured on a NanoReady ultra-micro UV-visible spectrophotometer and stored in a refrigerator at −20° C. for later use.

Finally, the basic fragments are obtained, and the adjacent basic fragments had 40-75 bp homologous sequences for recombination or fusion PCR, the fusion PCR is performed using adjacent 2-4 basic fragments as templates. The site UP (SEQ ID NO. 27), element GPMp (SEQ ID NO. 28), HcCYP81Q58, IDP1t (SEQ ID NO.29) are homologously recombined into a fragment, and the element TEF2p (SEQ ID NO. 30), HcCYP87D19, TDH2t (SEQ ID NO. 31) are homologously recombined into a fragment. The elements TEF1p (SEQ ID NO. 32), HcSDR34 (SEQ ID NO. 33), ENO2t (SEQ ID NO. 34), LEU2 (SEQ ID NO. 35), and site Down (SEQ ID NO. 36) are fused into a fragment by homologous recombination, each fusion fragment is constructed into a gene expression cassette (SEQ ID NO. 37).

The gene expression cassettes are ligated together by yeast homologous recombination and integrated into the chromosome 8 DNA site through yeast lithium acetate transformation (the detailed method steps of yeast homologous recombination, lithium acetate transformation, and integration, and positive bacteria identification have been recorded in the instructions of the inventor's previous patents CN202311356007.2 and CN202311746115.0). The primers used for each element are shown in Table 1 below.

TABLE 1
Amplification primers for constructing Saccharomyces cerevisiae
chassis cell strain

| Primers | Sequence ID NO. |
|---|---|
| UP-F | SEQ ID NO. 3 |
| UP-GPMP-R | SEQ ID NO. 4 |
| GPMP-UP-F | SEQ ID NO. 5 |
| GPMP-Hc81Q58-R | SEQ ID NO. 6 |
| Hc81Q58-GPMP-F | SEQ ID NO. 7 |
| Hc81Q58-IDP1t-R | SEQ ID NO. 8 |
| IDP1t-Hc81Q58-F | SEQ ID NO. 9 |
| IDP1t-TEF2p-R | SEQ ID NO. 10 |
| TEF2p-IDP1t-F | SEQ ID NO. 11 |
| TEF2p-Hc87D19-R | SEQ ID NO. 12 |
| Hc87D19-TEF2p-F | SEQ ID NO. 13 |
| Hc87D19-TDH2t-R | SEQ ID NO. 14 |
| TDH2t-Hc87D19-F | SEQ ID NO. 15 |
| TDH2t-ENO2t-R | SEQ ID NO. 16 |
| SDR-TEF1p-F | SEQ ID NO. 17 |
| TEF1p-SDR-R | SEQ ID NO. 18 |
| SDR-ENO2t-F | SEQ ID NO. 19 |
| SDR-TEF1p-R | SEQ ID NO. 20 |
| ENO2t-TDh2t-F | SEQ ID NO. 21 |
| ENO2t-SDR-R | SEQ ID NO. 22 |
| LEU2-TEF1p-F | SEQ ID NO. 23 |
| LEU2-DN-R | SEQ ID NO. 24 |
| DN-LEU2-F | SEQ ID NO. 25 |
| DN-R | SEQ ID NO. 26 |

### Embodiment 3: Construction and Expression of Saccharomyces cerevisiae Engineering Bacteria Coul-05-1

1. The host strain of Saccharomyces cerevisiae is transferred to YPD liquid medium (YPD liquid medium formula: 1% Yeast Extract (Yeast Extract (Angel Yeast Co., Ltd.)), 2% Peptone (peptone (Angel Yeast Co., Ltd.)), 2% Dextrose (dextrose)) for activation, and the activation conditions are 30° C., 220 rpm for 24 h; the method of culture in YPD liquid medium is as follows: the initial OD600 is 0.4; the Saccharomyces cerevisiae liquid is obtained after it is cultured at 30° C. and 220 rpm until OD600 reached 0.8-0.9;
2. The Saccharomyces cerevisiae liquid is transferred to the YPD liquid medium for culture, and the Saccharomyces cerevisiae bodies are collected;
3. 25 ml deionized water is taken to wash the Saccharomyces cerevisiae twice, the Saccharomyces cerevisiae precipitation is obtained after centrifuging at 5000 rpm for 5 min;
4. 1 ml 100 mM LiAc is added to resuspend the bacteria, and the bacterial precipitate is obtained after centrifuging at 8000 rp for 30 seconds and removing the supernatant.
5. The Saccharomyces cerevisiae precipitate is resuspended in 100 ul, 0.1 mM LiAc and packed in a 1.5 ml centrifuge tube, 240 ul PEG4000 solution, 36 ul LiAc solution, and 10 ul SSDNA are added, and the amount of DNA in each fragment is between 600-800 ng. The total system is supplemented with sterilized deionized water to 360 ul and mixed well, and it is cultured at 30° C. for 60 min, heat shock is maintained at 42° C. for 40 min, and 150 ul transformed bacterial solution is coated on the SC-LEU solid screening medium: 2% glucose, lacking the corresponding nutrient selection marker amino acid LEU; screening culture conditions: the medium is put upside down in 30° C. incubator for 2-3 days until the transformants grow out, a single colony is selected and put in the PCR tube, 10 ul 40 mm NaOH is added, and it is taken as a template after heating 98° C. for 10 min, PCR identification is carried out with primers in Table 2, and all the two corresponding target fragments are the correct positive clones, named as strain Coul-05-1.

TABLE 2
Primers used in PCR identification

| Primers | Sequence ID NO. |
|---|---|
| F1 | SEQ ID NO. 38 |
| R1 | SEQ ID NO. 39 |
| F2 | SEQ ID NO. 40 |
| R2 | SEQ ID NO. 41 |

TABLE 3
Saccharomyces cerevisiae strains used in this study

| Strains | Genotype or characteristic | Resource |
|---|---|---|
| BY4742 | MATα, his3Δ1, leu2Δ0, lysΔ0, ura3Δ0 | ATCC |
| DNHC87-03 | Downstream module (HXT7p-tHMG1-ADH1t, | Stored |
| | TEF2p-synHcCPR1-TDH2t, TPI1p-ERG1-ENO2t, | in the |
| | GPM1p-ERG20-CYC1t, PGK1p-ERG9-FBA1t, | laboratory |
| | TDH3p-synHcOSC6-PGT1t and LEU2 marker | |
| | gene) integrated into δDNA site of BY4742, | |
| | (UAS-TDH3p-synHcCYP87D20-PRM9t and | |
| | G418 marker gene) integrated into rDNA site of | |
| | BY4742. | |
| Cuol-05-1 | Upstream module (GPMp-HcCYP81Q58-IDP1t, | In this |
| | TEF2p-HcCYP87D19-TDH2t, TEF1p-HcSDR34- | study |
| | ENO2t and LEU2 marker gene) integrated into | |
| | YRPCδ15 DNA site of DNHC87-03. | |

### Embodiment 4: Identification of the Product Synthesized by Genetic Engineering Bacteria Coul-05-1

Fermentation of Saccharomyces cerevisiae engineering bacteria Cuol-05-1: Coul-05-1 fermentation seed liquid is prepared in YPD liquid medium (30° C., 200 rpm, 16 hours); the fermentation is transferred to a 250 ml flask containing 50 ml YPD liquid medium (30° C., 200 rpm, 6 h), and it is transferred to a 3 L flask containing 1LYPD liquid medium at 30° C., 200 rpm/min for expanded fermentation. The fermentation product is obtained by shaking culture for 8 days.

The conditions for extracting the product: the fermentation product was centrifuged at 8000 rpm for 10 min to collect the cell bacteria, and 300 ml of ethyl was used.

Extraction conditions: The fermentation product is centrifuged at 8000 rpm for 10 min to collect the cells, the cells are soaked with 300 ml ethyl acetate for 30 min, ultrasonically extracted for 30 min, and shaken once every 10 min. The cells are collected by centrifugation at 8000 rpm for 10 min, and the supernatant is taken to detect the product. The extracellular medium is extracted with an equal proportion of ethyl acetate, concentrated by a rotary evaporator, and isolated and purified by silica gel column chromatography.

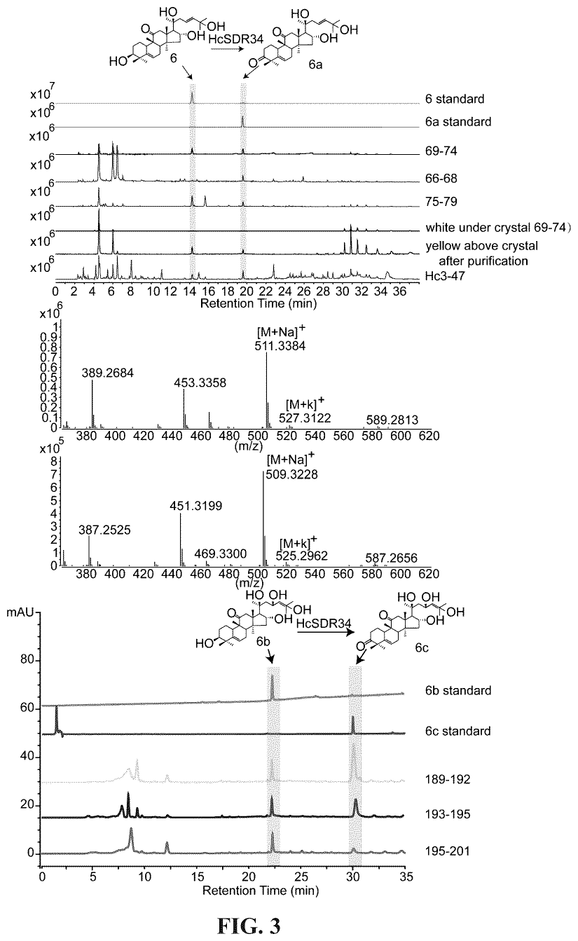

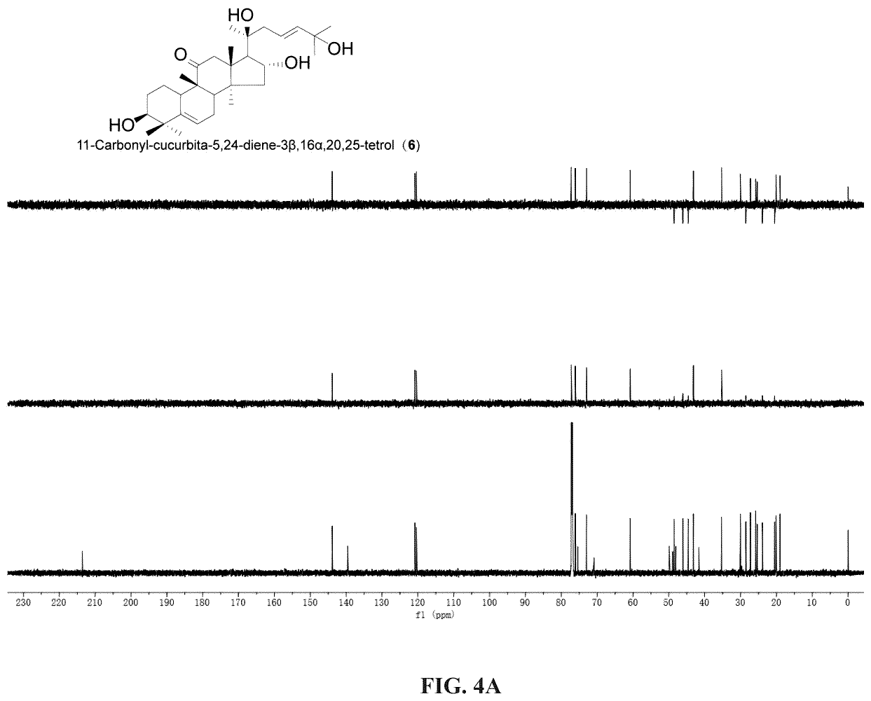

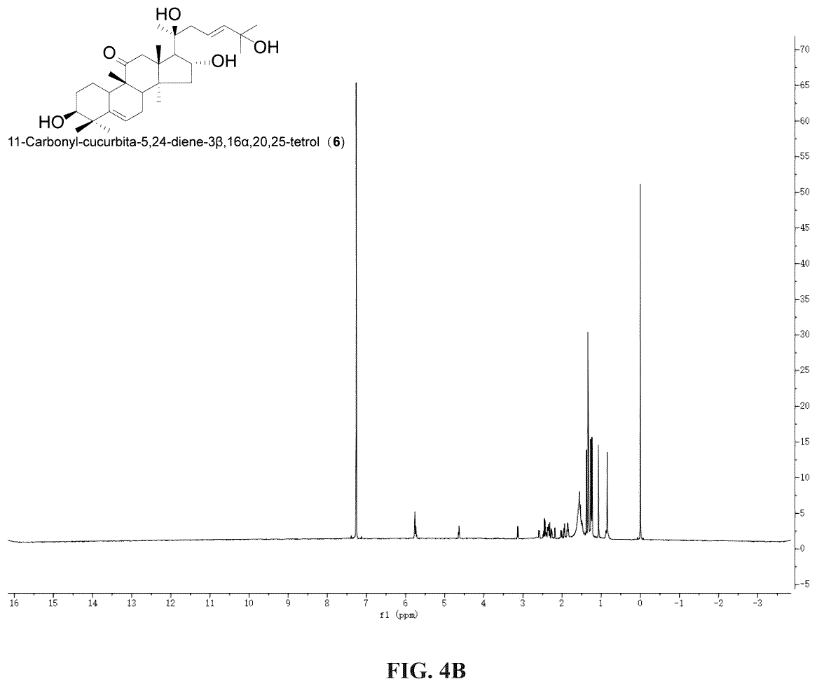

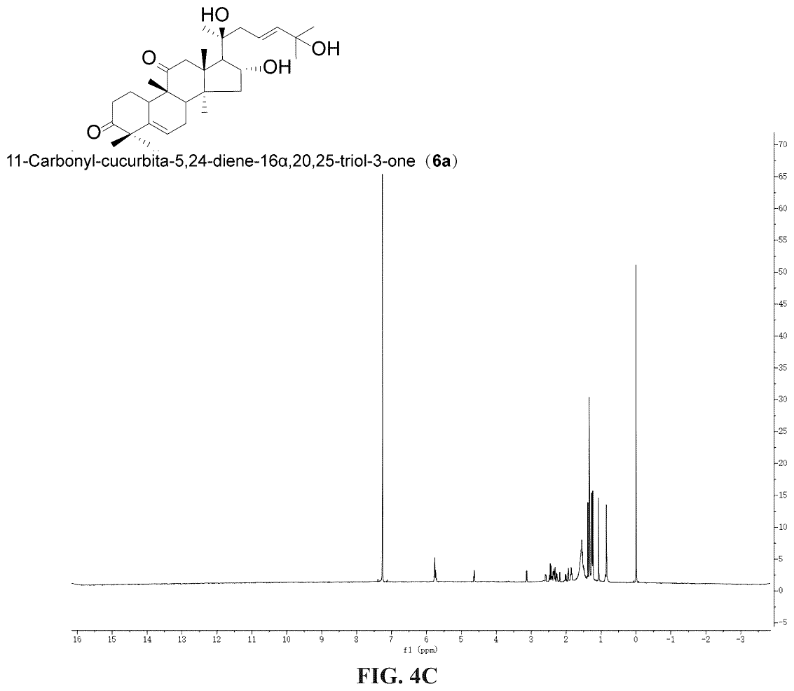

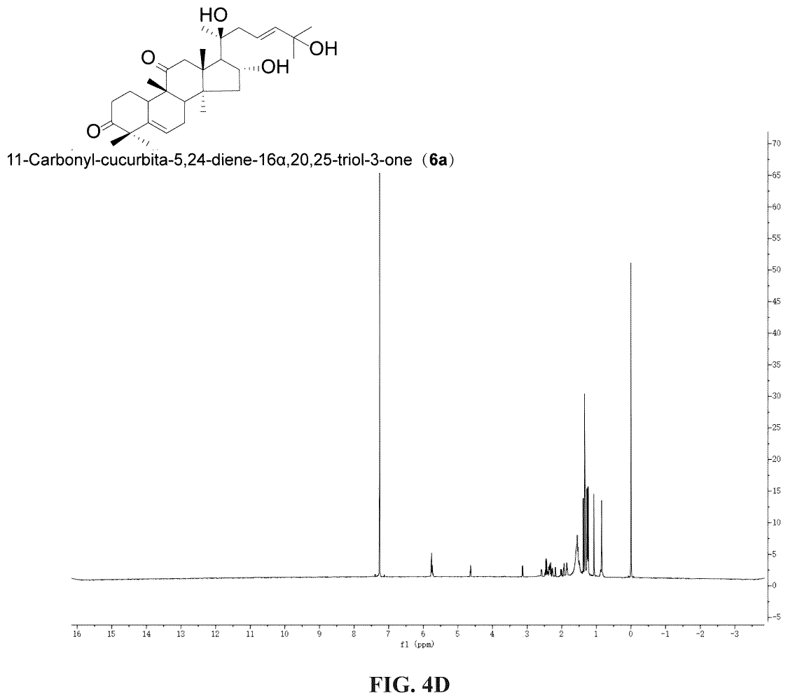

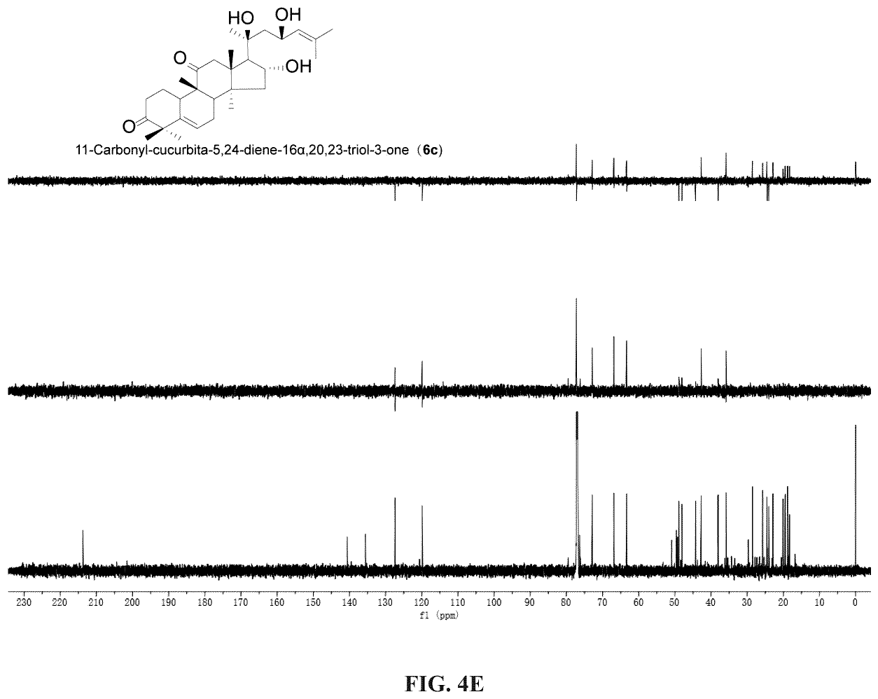

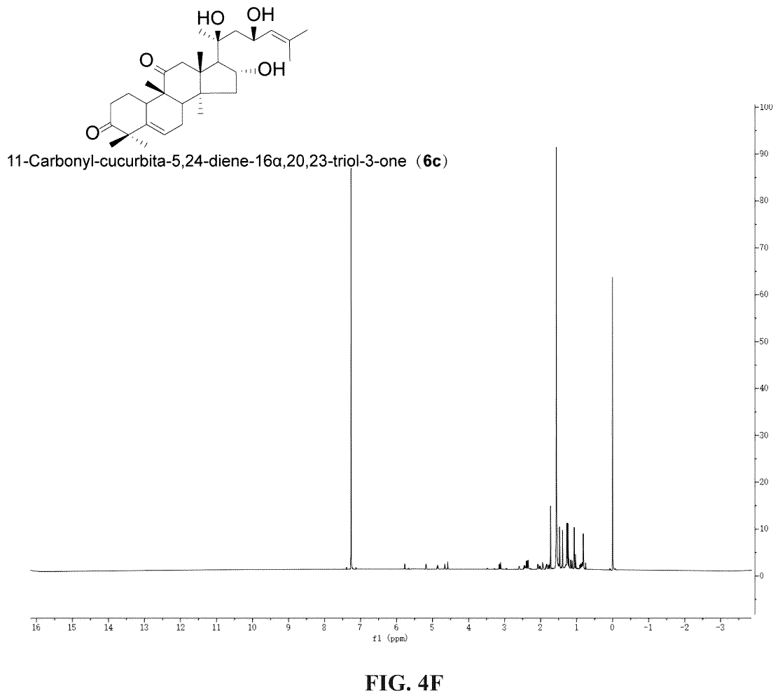

The fermentation products of Saccharomyces cerevisiae engineering bacteria Cuol-05-1 are identified by LC-Ms and NMR. The results are shown in Tables 4, 5 and FIGS. 3-4:

TABLE 4
13C&1H NMR (800 MHz, solvent: CDCl3) data
of HcCYP87D19 product (Jin Hz, δ in ppm)

| | δ_{H (1H)} | δ_{C (C13)} |
|---|---|---|
| Carbon(6) | | |
| 1 | 1.40 (m), 1.49 (m) | 20.52 |
| 2 | 1.67 (m), 1.78 (m) | 28.49 |
| 3 | 3.49(d, J = 3.4 Hz) | 76.09 |
| 4 | / | 41.62 |
| 5 | / | 139.54 |
| 6 | 5.68 (d, J = 5.8 Hz) | 120.38 |
| 7 | 1.94 (m), 2.41 (m) | 23.89 |
| 8 | 1.90 (d, J = 8.1 Hz) | 43.15 |
| 9 | / | 48.96 |
| 10 | 2.30 (m) | 35.28 |
| 11 | / | 213.56 |
| 12 | 2.38 (d, J = 14.2 Hz), | 48.5 |
| | 3.09 (d, J = 14.3 Hz) | |
| 13 | / | 48.07 |
| 14 | / | 49.92 |
| 15 | 1.51 (m), 1.84 (m) | 44.56 |
| 16 | 4.61 (t, J = 7.9 Hz) | 72.94 |
| 17 | 2.31 (m) | 60.78 |
| 18 | 0.82 (s, 3H) | 20.12 |
| 19 | 1.12 (s, 3H) | 20.07 |
| 20 | / | 75.4 |
| 21 | 1.32 (s, 3H) | 25.79 |
| 22 | 2.25 (m), 2.34 (m) | 46.07 |
| 23 | 5.73 (m) | 120.82 |
| 24 | 5.74(d, J = 819.4 Hz) | 143.86 |
| 25 | / | 70.84 |
| 26 | 1.33(s, 3H) | 29.98 |
| 27 | 1.33 (s, 3H) | 30.07 |
| 27 | 1.18 (s, 3H) | 25.34 |
| 29 | 1.03 (s, 3H) | 27.25 |
| 30 | 1.32 (s, 3H) | 18.98 |
| Carbon(6a) | | |
| 1 | 1.50 (m), 1.82 (m) | 24.46 |
| 2 | 2.35 (m), 2.45 (m) | 38.01 |
| 3 | / | 213.74 |
| 4 | / | 50.92 |
| 5 | / | 140.62 |
| 6 | 5.77 (m) | 119.86 |
| 7 | 2.03 (m), 2.40 (m) | 23.91 |
| 8 | 1.67 (d, J = 8.2 Hz) | 42.69 |
| 9 | / | 49.27 |
| 10 | 2.59 (d, J = 11.2 Hz) | 35.81 |
| 11 | / | 23.74 |
| 12 | 2.36 (m), 3.13 (m) | 48.88 |
| 13 | / | 49.6 |
| 14 | / | 49.54 |
| 15 | 1.54 (m), 1.83 (m) | 44.25 |
| 16 | 4.66 (t, J = 8.0 Hz) | 72.82 |
| 17 | 2.33 (m) | 63.33 |
| 18 | 0.81 (s, 3H) | 20.02 |
| 19 | 1.06 (s, 3H) | 19.47 |
| 20 | / | 76.13 |
| 21 | 1.48 (s, 3H) | 24.53 |
| 22 | 1.56 (m), 1.76 (m) | 48.03 |
| 23 | 4.86 (t, J = 9.8 Hz) | 66.86 |
| 24 | 5.18(d, J = 8.6 Hz) | 127.37 |
| 25 | / | 135.57 |
| 26 | 1.72 (s, 3H) | 18.24 |
| 27 | 1.72 (s, 3H) | 25.73 |
| 27 | 1.26 (s, 3H) | 22.84 |
| 29 | 1.23 (s, 3H) | 28.53 |
| 30 | 1.40 (s, 3H) | 18.79 |
| Carbon(6c) | | |
| 1 | 1.46 (m), 2.25 (m) | 25.65 |
| 2 | 1.61 (m), 1.96 (m) | 29.37 |
| 3 | 3.46 (m) | 76.67 |
| 4 | / | 42.24 |
| 5 | / | 142.2 |
| 6 | 5.57 (m) | 121.27 |
| 7 | 1.79 (m), 2.40 (m) | 24.32 |
| 8 | 1.67 (d, J = 7.4 Hz) | 43.26 |
| 9 | / | 39.97 |
| 10 | 2.47 (m) | 35.93 |
| 11 | 3.93(dd, J = 10.9, 6.0 Hz) | 79.04 |
| 12 | 1.79 (m), 1.82 (m) | 40.33 |
| 13 | / | 47.39 |
| 14 | / | 49.57 |
| 15 | 1.11 (m), 1.19 (m) | 34.33 |
| 16 | 1.29 (m), 1.90 (m) | 28.08 |
| 17 | 1.58 (m) | 50.08 |
| 18 | 0.89 (s, 3H) | 16.96 |
| 19 | 0.16 (s, 3H) | 25.91 |
| 20 | 1.51 (m) | 36.42 |
| 21 | 0.91(d, J = 6.4 Hz) | 18.84 |
| 22 | 1.72 (m), 2.14 (m) | 39.23 |
| 23 | 5.59(m) | 125.52 |
| 24 | 5.58 (m) | 139.75 |
| 25 | / | 70.96 |
| 26 | 1.31 (s, 3H) | 30.09 |
| 27 | 1.31 (s, 3H) | 30.18 |
| 27 | 1.04 (s, 3H) | 26.61 |
| 29 | 1.13 (s, 3H) | 25.99 |
| 30 | 0.82 (s, 3H) | 19.18 |

TABLE 5
Compound abbreviations and chemical
nomenclature of four products

| Abbreviation | Chemical name |
|---|---|
| 6 | 11-Carbonyl-cucurbita-5,23-diene- |
| | 3β,16α,20,25-tetrol |
| 6a | 11-Carbonyl-cucurbita-5,23-diene- |
| | 16α,20,25-triol-3-one |
| 6b | 11-Carbonyl-cucurbita-5,23-diene- |
| | 3β,16a,20,23-tetrol (not obtained) |
| 6c | 11-Carbonyl-cucurbita-5,24-diene- |
| | 16α,20,23-triol-3-one |

Although the embodiments of the invention have been shown and described above, it is understandable that the above embodiments are exemplary and cannot be understood as restrictions on the invention. Ordinary technicians in this field can change, modify, replace, and amend the above embodiments within the scope of protection of the invention.

What is claimed is:

1. A Hemsleya chinensis cytochrome oxidase HcCYP87D19, a sequence of the gene is shown in SEQ NO: 1.

2. An application of the Hemsleya chinensis cytochrome oxidase HcCYP87D19 according to claim 1 in a preparation of cucurbitacin intermediates.

3. The application according to claim 2, wherein the application is to co-transfect the cytochrome oxidase HcCYP87D19 and a HCCYP81Q58 into genetic engineering bacteria.

4. The application according to claim 2, wherein the cucurbitacin intermediates comprise 11-Carbonyl-cucurbita-5,23-diene-3β,16a,20,25-tetrol(6), 11-Carbonyl-cucurbita-5,23-diene-3β,16α,20,23-tetrol(6b), 11-Carbonyl-cucurbita-5,23-diene-16α,20,25-triol-3-one(6a), 11-Carbonyl-cucurbita-5,24-diene-16α,20,23-triol-3-one(6c).

5. An expression vector, which contains a promoter, HcCYP81Q58, HcCYP87D19, HcSDR34, LEU2, and a terminator.

6. The expression vector according to claim 5, wherein the promoter consists of GPMp, TEF2p, and TEF1p elements; the terminator consists of IDP1t, TDH2t and ENO2t, elements.

7. Transgenic Saccharomyces cerevisiae engineering bacteria, wherein the transgenic Saccharomyces cerevisiae engineering bacteria express expression vector according to claim 5.

8. A method for constructing the transgenic Saccharomyces cerevisiae engineering bacteria according to claim 7, wherein the method comprises transfecting the expression vector according to claim 5 into Saccharomyces cerevisiae.

9. An application of the Hemsleya chinensis cytochrome oxidase HcCYP87D19 gene according to claim 1 in a preparation of cucurbitacin intermediate products.

10. The method according to claim 8 in a preparation of cucurbitacin intermediates.

11. Transgenic Saccharomyces cerevisiae engineering bacteria, wherein the transgenic Saccharomyces cerevisiae engineering bacteria express expression vector according to claim 6.

12. A method for constructing the transgenic Saccharomyces cerevisiae engineering bacteria according to claim 7, wherein the method comprises transfecting the expression vector according to claim 6 into Saccharomyces cerevisiae.

13. An application of the expression vector according to claim 5 in a preparation of cucurbitacin intermediate products.

14. An application of the expression vector according to claim 6 in a preparation of cucurbitacin intermediate products.

15. An application of the transgenic Saccharomyces cerevisiae engineering bacteria according to claim 7 in a preparation of cucurbitacin intermediate products.
